(19) United States
(12) Patent Application Publication
(10) Pub. No.: US 2010/0042138 A1
(43) Pub. Date: Feb. 18, 2010

(54) ANTI-MIGRAINE DEVICE

(76) Inventor: Juan José Duelo Riu, Barcelona (ES)

Correspondence Address: HUGHES HUBBARD & REED LLP, ONE BATTERY PARK PLAZA, NEW YORK, NY, 10004, US

(21) Appl. No.: 12/521,925

(22) PCT Filed: Oct. 26, 2007
(86) PCT No.: PCT/ES2007/000610

§ 371 Date: Jul. 1, 2009

(30) Foreign Application Priority Data
Feb. 8, 2007 (ES) U 200700264

(51) Int. Cl. A61B 17/00

(52) U.S. Cl. 606/204

(57) ABSTRACT

This device is formed by a number of semi-rings and their extensions, adapted for fitting to the human head of a patient and for exerting pressure on predetermined parts of said head, comprising a principal open crown (12) configured in the shape of a “U” formed by a slightly concave back zone (13), which extends in two side zones (14-15) converging inwards, and whose extremities or sidepieces (16-6′) have on the inside thereof cavities (19-19′) adapted for housing contact elements (17-17′), wherein the front surface is perceptibly warped convexly, partly projecting inwards inside the crown (12). The extremities or sidepieces (16-6′) have in upper portion thereof cavities (18-18′) directed inwards with a certain inclination with respect to the plane of the principal crown (12) adapted for housing the ends of a second open crown (11).

The present invention refers, in accordance with the above title of the description, to an “anti-migraine device”, whose new characteristics of construction, shape and design comply with the regulations while obtaining maximum safety and effectiveness.

More specifically the invention refers to a crown-like device that is open at the front, the ends of which are connected to an auxiliary ring that is open at the ends, and where said crown is fitted with some contact elements that curve inwardly at their two ends. The device is to be fitted to the head of a patient and the aim is to exercise pressure in specific parts of the head, the temples, mainly the parietal areas so as to lessen the pain produced by migraine.

Migraine manifests itself with intermittent pain to a part of the human head and there is no specific treatment and according to the statistics 16% of the population suffers from it. Up to now neurologists have identified more than 165 types of headache. The sensation of pain is manifested in the cerebral periphery and during the migraine crisis certain veins and arteries that irrigate the brain show blood vessel dilation caused by an inflammation of a non-infectious cause. When there is a gushing of blood through the irrigated area, it causes a pressure on the dilated blood vessel that converts into intense pain. The pain is manifested in the temporal arteries that can be felt on the temples. The majority of people affected by migraine maintain that instinctively when the pain appears they press their fingers onto this area and in this way the pain is eased. This device has been designed on the basis of this symptomatology that enables the pressure to be applied to the temples and deadens the blow felt by the blood passing through the dilated artery.

An “anti-migraine band” by the same owners is known under Spanish Invention Patent No 9702519, which in accordance with its first claim it is characterised in that its pressure terminals or surfaces are under the action of some means to regulate the closeness to the human head and some means to regulate the curvature, that act on the eccentricity of the pressure surface. Said band has undergone numerous modifications over time which has resulted in the aim of this present invention that, starting from the object of said patent, presents structural characteristics that are completely new.

The improvements of the invention are based on the new structure which is characterised by having two crowns open at the front; the principal crown that makes the body of the band has an ergonomic arrangement with an irregular section that adapts perfectly to shape of the head of the patient and is made of a flexible plastic material (by preference polycarbonate lexan). This flexible plastic material has great resistance to deformation and therefore has ideal resistance to the opening and closing of the ends when the band is put on and taken off of the head by the user.

This principal crown includes an element that makes contact with the temple of the patient that is fitted close to each end of the two longer parts. Said contact elements have projections with a slightly elongated asymmetrical convex shape, in such a way that the surface of these projections makes contact with the parietal areas of the patient in a broad area for the purpose of improving the treatment of all types of migraines. These contact elements are made of an elastomer plastic, by preference satoprene shore 40, which allows for a pleasant and relaxing contact on the skin of the patient in the area to be treated.

Some cavities have been planned, the perimeter arrangement of which coincides with the contact elements for said end parts of the principal crown, and for the purpose of housing the contact elements. The fixing of these contact elements into these cavities can be by pressure, with the aid of an adhesive material, or other equivalent means.

Optionally a second open crown can be provided, formed by a metallic stay with a smaller section than the principal crown, converges close to the ends of the principal crown. This second crown is fixed to the principal crown thanks to some cavities made in the principal crown adapted so as to house the ends of the said second crown by pressure.

The purpose of this stay is to allow a constant intensity of pressure to be maintained by the device, the ends of the second crown imposing a force on the ends of the first, and its use is appropriate in those cases in which the migraine of the patient is very severe. Additionally, this second crown allows for a better adjustment of the device object of the invention, as he human head has different dimensions a single open crown would not be sufficient in some cases for the correct positioning of said device.

In an alternative embodiment it has been envisaged that the contact elements are removable and interchangeable by some suitable means for temporary fixing in the cavities.

These removable elements include or are made up from a substance that is suitable for cooling, such as a gel or something similar that can be frozen, thus allowing for the application of cold with the pressure. In this way the user can freeze just the contact elements until the moment that the device is used, so as to achieve greater relief with the cold.

It has also been envisaged that the sidepieces have a variable length, thus being able to adjust the position of the contact elements onto the appropriate point on the head of the patient. This regulation can be achieved by some telescopic devices, for example a rod housed in a respective hollow guide.

Other details and characteristics will be made clear in the description that is given below, in which reference is made to the drawings that are attached to this description and which is made by way of illustration on the practical embodiment of the invention, but without being by way of being a limitation.

In said drawings:

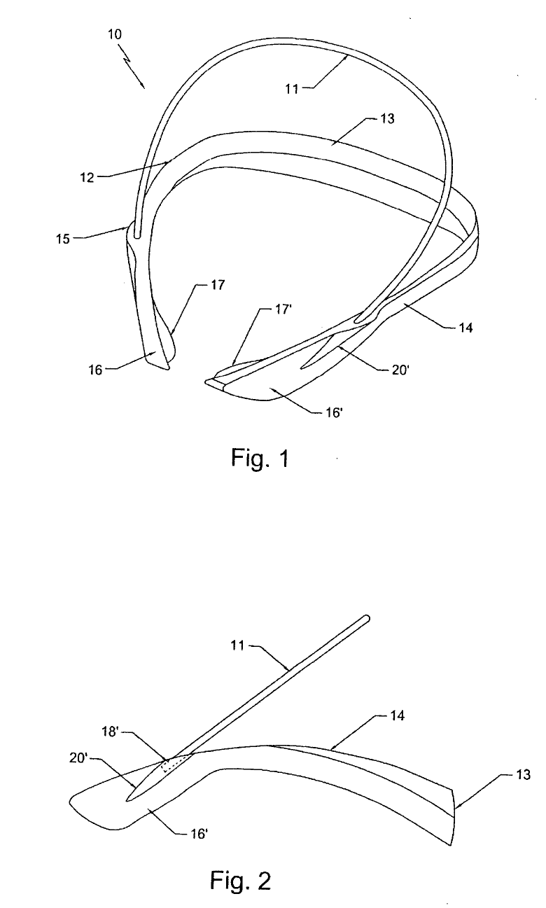

FIG. 1 is a perspective view of the device object of the invention, which is formed by the joining of two crowns open at the ends in conjunction with some contact elements.

FIG. 2 is a side elevated view of the device object of the invention, where the sidepieces are shown, that are fitted to the end areas of the principal crown, and also the side areas.

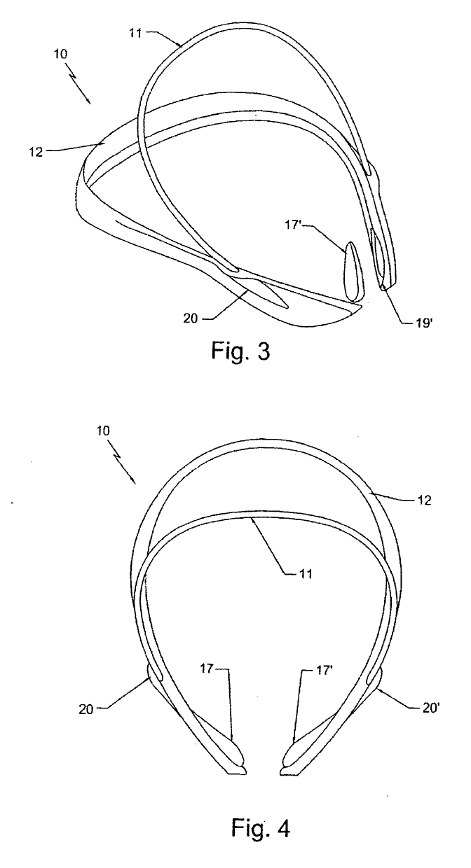

FIG. 3 is a perspective view of the device object of the invention, in which one of the two cavities can be seen for the housing of the contact projections.

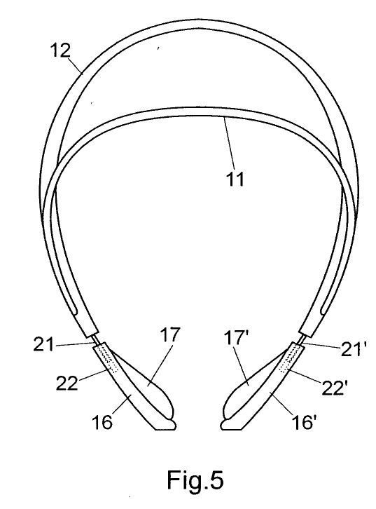

FIG. 4 is a top plan view of the device object of the invention.

FIG. 5 is a top plan view of the device object of the invention with a variation with sidepieces that can have their length regulated in a telescopic manner.

Below a numerical list follows of the main elements of the invention and which is illustrated in the stated attached figures; (10) device, (11) open crown, (12) principal open crown, (13) rear area of (12), (14-15) side areas of (12), (16-16′) sidepieces, (17-17′) contact elements, (18-18′) cavities, (19-19′) cavities, (20-20′) projections, y (21-21′) rods with telescopic means housed in (22-22′) hollow guides in the sidepieces (16, 16′).

In one of the preferred embodiments of that which is the aim of this present invention, the device (10) includes a principal crown (12) with an appreciably open oval arrangement, and rod (11) with a regular section that is assembled by its ends on the end part of the principal crown (12) forming a “U” shape, see FIGS. 1, 3 and 4.

The principal crown (12) is made from by a material body that by preference is a flexible plastic, with a slightly concave rear area (13), that extends in both side areas (14-15) that converge inwards, and whose ends or sidepieces (16-16′) have some cavities (19-19′) on the front inside part adapted so as to house some contact elements (17-17′), the front surface of which has an appreciably warped convex shape, which once introduced into said cavities (19-19′) partially overlaps inwardly into the crown (12). These contact elements (17-17′) are manufactured from a plastic elastomer so as to contribute towards patient comfort. It has also been envisaged that these contact elements (17-17′) include a substance in the form of a cryogenic gel so as to be chilled and applied cold onto the contact area of the patient's head. In order to do this it has been planned that the coupling of said contact elements (17-17′) is carried out in a manner that is completely removable and interchangeable, by means of temporary connection devices.

The height of the principal crown (12) is irregular, being higher at the rear (13) than at the side areas (14-15), and then once again widening out in the part of the sidepieces (16-16′). On the other hand, the upper edge of the part of the sidepieces (16-16′) is slightly wider than the rest of the principal crown (12) so as to be able to house the cavities (18-18′) and (19-19′), see FIG. 4.

The principal crown (12) shape converges in the shape of a “U” and is perfectly adapted to the curve of the head of the patient, and likewise the inner contact surface of the all of the principal crown (12) with the head having a slight concave curve that guarantees the perimeter support of the principal crown (12) on the middle area of the patient's head, see FIG. 2. In addition, the side areas (14-15) of the principal crown (12) have a profile with a slight concave curve that extends in the sidepieces (16-16′), so that it can adjust with greater comfort to the mid area of the patient's head, see FIG. 2.

The second crown (11), as and how seen in FIGS. 1, 3 and 4 is made up with releasable metallic rods with a small diameter (approximately 3 mm.), the ends of which are inserted by pressure into the cavities (18-18′) made in the body of the sidepieces (16) that are found in the ends of the principal crown (12). These cavities (18-18′) are formed in the surface or upper edge of the sidepieces (16-16′) and are aimed inwards with a certain angle, by preference 20°-60°, compared to the plane of the crown (12). Because the thickness of the sidepieces is so small, the outer surface of the sidepieces (16-16′) is fitted with a projection that overlaps outwards (20-20′) so as to house the stated cavities (18-18′).

This means that as the second crown (11) is assembled it makes a certain angle against the principal crown so that it can be supported on the mid-high area of the patient's head, see FIG. 2.

The purpose of said second crown (11) is to press the sidepieces (16) of the principal crown (12) inwards, this means to converge on each other (16), reinforcing the converging action caused from the shape of the principal crown (12) and exercising a more constant intensity of pressure on the patient by means of the contact elements (17-17′).

The joint use of the two open crowns (11) and (12), allows in addition to adding the actions of pressure inwards of the end parts, that of the improving of the positioning of the device (10) on the head of the user, and in so doing preventing the anti-migraine device (10) from being able to slip or move on its own.

An alternative embodiment can be seen in FIG. 5, in which both sidepieces (16-16′) have some telescopic means of adjusting their lengths, allowing the position of the contact elements (17-17′) to be regulated. These telescopic devices are made from a set of rods (21-21′) and hollow guides (22-22′).

Having sufficiently described this present invention in combination with the attached figures it is easy to see that any modifications to the detail that are considered advisable can be introduced, provided that they do not alter the essence of the invention that is summarised in the following claims.

1. An anti-migraine device that is made up by semi-rings and their extensions and adapted for fitting to the head of a patient and for exerting pressure on predetermined parts of the head, characterised in that it comprises a principal open crown (12) configured in the shape of a “U” formed by a slightly concave back zone (13), which extends in two side zones (14-15) that converge inwards and whose extremities or sidepieces (16-16′) have on the inside thereof cavities (19-19′) adapted for housing contact elements (17-17′), wherein the front surface is perceptibly warped convexly, which once introduced into the contact elements (17-17′) of said cavities (19-19) partially overlap inwardly into the crown (12), and whose extremities or sidepieces (16-16′) have in upper portion thereof cavities (18-18′) directed inwards with a certain inclination with respect to the plane of the principal crown (12) adapted for housing the ends of the second open crown (11).

2. Anti-migraine device according to claim 1, characterised in that the inclination of the cavities (18-18′) for the second open crown (11) with respect to the plane of the principal crown (12) is between 20° and 60°.

3. Anti-migraine device according to claim 1, characterised in that the outer surface of the sidepieces (16-16′) has a projection (20-20′) overlapping outwards in order to house the cavities (18-18′) for the second open crown (11).

4. Anti-migraine device according to claim 1, characterised in that the height of the principal crown (12) is irregular, being higher at the rear (13) than at the side areas (14-15), and then once again widening out in the part of the sidepieces (16-16′).

5. Anti-migraine device according to claim 1, characterised in that the inner contact surface of all of the principal crown (12) with the head of the patient has a slight concave curve, adapted for the perimeter support of the principal crown (12) in the mid area of the head.

6. Anti-migraine device according to claim 1, characterised in that on the side areas (14-15) of the principal crown (12) there is a profile with a slight concave curvature that extends into the sidepieces (16-16′).

7. Anti-migraine device according to all of the above claims, characterised in that the principal crown (12) is made from a flexible plastic material.

8. Anti-migraine device according to claim 1, characterised in that the contact elements (17-17′) that are inserted into the cavities (18-18′) of the principal crown (12) are made from a plastic elastomer.

9. Anti-migraine device according to claim 1, characterised in that the second crown (11) is releasable and on assembly has an appreciably concave arrangement, its ends being inserted by pressure in some cavities (18-18′) fitted in the sidepieces (16-16′) of the principal crown (12), and being adapted so that it can be fitted and supported on the mid-high area of the head of the patient.

10. Anti-migraine device according to claims 1 and 9, characterised in that the second open crown (11) is made out of a small diameter metallic rod.

11. Anti-migraine device according to claim 1, characterised in that the contact elements (17-17′) are removable and changeable by means of some suitable means of temporary fixing into the cavities (19-19′).

12. Anti-migraine device according to claim 11, characterised in that the contact elements (17-17′) include a substance, a gel or something similar, that is suitable for chilling for the application of cold onto the contact point of the patient's head.

13. Anti-migraine device according to claim 1, characterised in that the sidepieces (16-16′) have a variable length.

14. Anti-migraine device according to claim 13, characterised in that the sidepieces (16-16′) have some telescopic means of adjustment with at least a rod housed in a hollow guide.
